(19) United States
(12) Patent Application Publication
(10) Pub. No.: US 2007/0004724 A1
(43) Pub. Date: Jan. 4, 2007

(54) (+)-(2S,3s)-2-(3-chlorophenyl)-3,5,5-trimethyl-2-morpholinor for treating anxiety

(76) Inventors: Judith Bramel Deely, Durham, NC (US); Philip Alan Gerrard, Verona (IT); Elizabeth Parsons Goodale, Durham, NC (US); Susan Marie Learned-Coughlin, Durham, NC (US); Malcolm Stuart Nobbs, Harlow, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,916

(22) PCT Filed: Nov. 25, 2004
(86) PCT No.: PCT/EP04/13499

§ 371 Date: May 19, 2006

(30) Foreign Application Priority Data
Nov. 27, 2003 (GB) 0327619.3

(51) Int. Cl. A61K 31/5375

(52) U.S. Cl. 514/237.5

(57) ABSTRACT

This invention relates to a novel method of treatment employing (+)-(2S,3S)-2-(3-chlorophenyl)-3,5,5-trimethyl-2-morpholinol hydrochloride or a salt or solvate thereof, in particular its use in the treatment of anxiety disorders or in the treatment of mixed anxiety-depressive disorder.

This invention relates to a novel use of (+)-(2S,3S)-2-(3-chlorophenyl)-3,5,5-trimethyl-2-morpholinol, in particular its use in the treatment of anxiety disorders, or its use in the treatment of mixed anxiety-depressive disorder.

### BACKGROUND OF THE INVENTION

Bupropion hydrochloride, (±)-1-(3-chlorophenyl)-2-[(1,1-dimethylethyl)-amino]-1-propanone hydrochloride, is the active ingredient of Wellbutrin® which is marketed in the United States for the treatment of depression. It is also the active ingredient of Zyban® which is marketed in the United States as an aid to smoking cessation. Bupropion is an inhibitor of the neuronal uptake of noradrenaline (NA), and dopamine (DA), does not inhibit monoamine oxidase and has a negligible effect on the neuronal uptake of seretonin. While the mechanism of action of bupropion, as with other antidepressants, is not fully understood, it is presumed that this action is mediated by noradrenergic and/or dopaminergic mechanisms. Initial clinical evidence suggested Wellbutrin® to be a selective inhibitor of noradrenaline (NA) at doses that are predictive of antidepressant activity in animal models (Ascher, J. A., et al., Journal of Clinical Psychiatry, 56: p. 395-401, 1995). A more recent analysis (Stahl, S. M. et al., Prim. Care Companion, Journal of Clinical Psychiatry, 6(4), p 159-166, 2004) concludes that bupropion acts via dual inhibition of norepinephrine and dopamine reuptake, having slightly greater functional potency at the dopamine transporter.

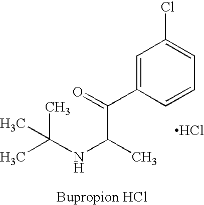

Bupropion is extensively metabolized in man as well as laboratory animals. Urinary and plasma metabolites include biotransformation products formed via hydroxylation of the tert-butyl group and/or reduction of the carbonyl group of bupropion. Four basic metabolites have been identified. They are the erythro- and threo-amino alcohols of bupropion, the erythro-amino diol of bupropion (found in urine but not in plasma), and a morpholinol metabolite.

The morpholinol metabolite (+/−)-(2R*,3R*)-2-(3-chlorophenyl)-3,5,5-trimethyl-2-morpholinol is believed to be formed from hydroxylation of the tert-butyl group of bupropion.

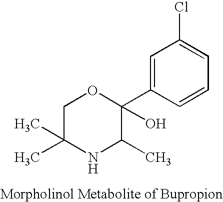

It was discovered that despite the (−) form of the morpholinol metabolite predominating significantly in human plasma samples, it was the (+) enantiomer, (+)-(2S,3S)-2-(3-chlorophenyl)-3,5,5-trimethyl-2-morpholinol in which the optimal monoamine reuptake inhibitory activity resides (WO 99/37305), hereinafter referred to as the compound of formula (I):

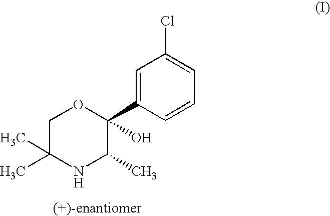

The compound of formula (I) and its salts and solvates have been disclosed as being of use in the treatment of depression (including major depressive disorder (MDD), bipolar depression (type I and II), major (unipolar) depression and depression with atypical features (eg. lethargy, over-eating/obesity, hypersomnia)), attention deficit hyperactivity disorder (ADHD), obesity, migraine, pain (including neuropathic pain, eg. diabetic neuropathy, sciatica, non-specific lower back pain, multiple sclerosis pain, fibromyalgia, HIV-related neuropathy, neuralgia such as as post-herpetic neuralgia and trigeminal neuralgia and pain resulting from physical trauma, amputation, cancer, toxins or chronic inflammatory conditions), sexual dysfunction (including inhibited sexual desire (low libido), inhibited sexual arousal or excitement, orgasm dysfunction, inhibited female orgasm, inhibited male orgasm, hypoactive sexual desire disorder (HSDD), female sexual desire disorder (FSDD) and sexual dysfunction side-effects induced by treatment with antidepressants of the SSRI-class), Parkinson's disease (including relief from the symptoms of Parkinson's disease which include, but are not limited to, locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), Alzheimer's disease, or addiction to cocaine or nicotine-containing (especially tobacco) products (WO 99/37305 and US2003-0064988; both Glaxo Group Limited).

US2003-0032643 (Glaxo Group Limited) discloses the use of the compound of formula (I) and its salts and solvates in the treatment of seasonal affective disorder, chronic fatigue, narcolepsy and cognitive impairment.

US2003-0083330 (Glaxo Group Limited) discloses the use of the compound of formula (I) and its salts and solvates in the treatment of addiction to alcohol.

WO 00/51546 and WO 01/62257 (both Sepracor Inc.) disclose the use of a bupropion metabolite in the treatment of a disorder that is ameliorated by the inhibition of neuronal monoamine reuptake, sexual dysfunction (including erectile dysfunction), an affective disorder (including depression, anxiety disorders, attention deficit hyperactivity disorder, bipolar and manic conditions, sexual dysfunction, psycho-sexual dysfunction, bulimia, obesity or weight gain, narcolepsy, chronic fatigue syndrome, seasonal affective disorder, premenstrual syndrome, and substance addiction or abuse), nicotine addicton, a cerebral function disorder (including senile dementia, Alzheimer's type dementia, memory loss, amnesia/amnestic syndrome, epilepsy, disturbances or consciousness, coma, lowering of attention, speech disorders, Parkinson's disease, Lennox syndrome, autistic disorder, autism, hyperkinetic syndrome, schizophrenia, cerebral infarction, cerebral bleeding, cerebral auteriosclerosis, cerebral venous thrombosis and head injury), epilepsy, smoking cessation and incontinence.

### SUMMARY OF THE INVENTION

The present invention provides the use of (+)-(2S,3S)-2-(3-chlorophenyl)-3,5,5-trimethyl-2-morpholinol or a salt or solvate thereof, or pharmaceutical compositions thereof, in the manufacture of a medicament for the treatment of anxiety disorders.

A further aspect of the invention provides the use of (+)-(2S,3S)-2-(3-chlorophenyl)-3,5,5-trimethyl-2-morpholinol or a salt or solvate thereof, or pharmaceutical compositions thereof, in the manufacture of a medicament for the treatment of mixed anxiety-depressive disorder.

A further aspect of the invention provides a method of treating anxiety disorders in a mammalian (human or animal) subject comprising the administration to said subject of an effective amount of (+)-(2S,3S)-2-(3-chlorophenyl)-3,5,5-trimethyl-2-morpholinol or a salt or solvate thereof, or pharmaceutical compositions thereof.

A further aspect of the invention provides a method of treating mixed anxiety-depressive disorder in a mammalian (human or animal) subject comprising the administration to said subject of an effective amount of (+)-(2S,3S)-2-(3-chlorophenyl)-3,5,5-trimethyl-2-morpholinol or a salt or solvate thereof, or pharmaceutical compositions thereof.

A further aspect of the present invention provides the use of enantiomerically pure (+)-(2S,3S)-2-(3-chlorophenyl)-3,5,5-trimethyl-2-morpholinol or a salt or solvate thereof or pharmaceutical compositions thereof in the manufacture of a medicament for the treatment of anxiety disorders.

A further aspect of the present invention provides the use of enantiomerically pure (+)-(2S,3S)-2-(3-chlorophenyl)-3,5,5-trimethyl-2-morpholinol or a salt or solvate thereof or pharmaceutical compositions thereof in the manufacture of a medicament for the treatment of mixed anxiety-depressive disorder.

A further aspect of the invention provides a method of treating anxiety disorders in a mammalian (human or animal) subject comprising the administration to said subject of an effective amount of enantiomerically pure (+)-(2S,3S)-2-(3-chlorophenyl)-3,5,5-trimethyl-2-morpholinol or a salt or solvate thereof, or pharmaceutical compositions thereof.

A further aspect of the invention provides a method of treating mixed anxiety-depressive disorder in a mammalian (human or animal) subject comprising the administration to said subject of an effective amount of enantiomerically pure (+)-(2S,3S)-2-(3-chlorophenyl)-3,5,5-trimethyl-2-morpholinol or a salt or solvate thereof, or pharmaceutical compositions thereof.

### DETAILED DESCRIPTION OF THE INVENTION

It will be appreciated that references herein to “treatment” extend to prophylaxis, prevention of recurrence and suppression or amelioration of symptoms (whether mild, moderate or severe) as well as the treatment of established conditions.

As used herein, the term “treatment of anxiety disorders” includes the treatment of generalised anxiety disorders, panic disorder with agoraphobia, panic disorder without agoraphobia, agoraphobia without a history of panic disorder, phobic disorders including social phobias (for example, social anxiety disorders) and specific phobias (also known as simple phobias), obsessive-compulsive disorder, stress disorders (including acute stress disorders and post-traumatic stress disorders), separation anxiety, adjustment disorders with anxious features, anxiety disorders due to a general medical condition, and anxiety disorders due to substance abuse.

Preferred is the treatment of generalised anxiety disorders, panic disorder (with or without agoraphobia), social phobias (for example social anxiety disorders), or stress disorders (including acute stress disorders and post-traumatic stress disorders). Most preferred is the treatment of generalised anxiety disorders (GAD), panic disorder, social anxiety disorders (SAD), or post traumatic stress disorders (PTSD). Of particular note in the context of the present invention is the treatment of generalised anxiety disorders (GAD), or the treatment of social anxiety disorders (SAD).

As used herein, the term “treatment of mixed anxiety-depressive disorder” refers to the treatment of such a disorder as defined in the appendix of the Diagnostic and Statistical Manual of Mental Disorders, Fourth Edition, 1994 (DSM-IV). The diagnostic criteria for this disorder generally include the presence of persistent or recurrent dysphoric mood lasting for ≧4 weeks and accompanied by ≧4 of the following symptoms: concentration or memory difficulties, sleep disturbances, fatigue or low energy, irritability, worry, being easily moved to tears, hypervigilence, anticipating the worst, hopelessness or pessimism about the future, or low self-esteem or feelings of worthlessness. The diagnosis encompasses subjects who do not meet the diagnostic criteria for either a depressive disorder or an anxiety disorder, but display a combination of non-specific anxious and depressive symptoms.

As used herein, “enantiomerically pure” means that the composition contains greater than about 90% of the desired stereoisomer by weight, preferably greater than about 95% of the desired stereoisomer by weight, more preferably greater than about 99% of the desired enantiomer by weight, most preferably greater than 99.5% of the desired enantiomer by weight, said weight percent based upon the total weight of the compound of formula (I).

Preferred for use according to the present invention are pharmaceutically acceptable salts or solvates of the compound of formula (I), particularly those disclosed in U.S. Pat. No. 6,342,496 B1, U.S. Pat. No. 6,337,328 B1, U.S. Pat. No. 6,391,875 B1, U.S. Pat. No. 6,274,579 B1, U.S. Patent Application Publication Nos. 2002/0052340 A1, 2002/0052341 A1, and 2003/0027827 A1, as well as WO 01/62257, WO 99/37305, WO 00/51546 and WO 01/62257. Suitable pharmaceutically acceptable salts can include, but are not limited to, hydrochloride salt, hydrogen sulfate salt and other sulfate salts, hydrogen phosphate salt and other phosphate salts, methanesulfonate salt, p-toluenesulfonate salt, citrate salt, fumarate salt, tartrate salt, and the like. Of these, (+)-(2S,3S)-2-(3-chlorophenyl)-3,5,5-trimethyl-2-morpholinol hydrochloride is particularly preferred.

Anxiolytics in current clinical use include selective serotonin re-uptake inhibitors (SSRIs) and benzodiazepines. There are no compounds approved for the treatment of anxiety disorders that are either noradrenaline or dopamine reuptake inhibitors. The results of the studies below illustrate that the compound of formula (I) may have a unique anxiolytic profile. In particular, the acute rat social interaction study suggests a different profile from that of SSRIs, in that there is a lack of anxiogenic activity for the compound of formula (I). The evidence of anxiolytic activity from all three studies described below, together with the absence of anxiogenic activity suggests that the compound of formula (I) may have an advantageous anxiolytic profile.

### DESCRIPTION OF THE DRAWINGS

FIG. 1. Effect of compounds on rat social interaction (Male Sprague Dawley rats, 350-400 g, n=10-14); chronic test

FIG. 2. Effect of compounds on rat social interaction (Male Sprague Dawley rats, 350-400 g, n=10-14); acute test

FIG. 3. Effect of compounds on mice in the light & dark box (CD-1 mice, 25-30 g, n=8-10)

### PREPARATION

The compound of formula (I) or a salt or solvate thereof may be prepared in isolated form, and preferably in an enatiomerically pure form, in accordance with the procedures set forth in WO 99/37305, US2003-0064988, US2003-0032643 and US2003-0027827 (all of Glaxo Group Limited) or WO 00/51546 and WO 01/62257 (both of Sepracor Inc.) the procedures of which are herein incorporated by reference.

### Dosage and Formulation

The compound of formula (I) or a salt or solvate thereof is administered in isolated form, and is preferably administered in an enantiomerically pure form.

The amount of compound of formula (I) or a salt or solvate thereof required to achieve the desired therapeutic effect will, of course depend on a number of factors, for example, the mode of administration and the recipient being treated in general, the daily dose will be in the range of 0.02 to 5.0 mg/kg, more particularly 0.1 to 1.5 mg/kg, and 0.15 to 1.2 mg/kg, and 0.3 to 1.2 mg/kg, and 0.3 to 2.4 mg/kg. More particular ranges include 0.02 to 2.5 mg/kg, 0.02 to 1.0 mg/kg, and 0.1 to 1.5 mg/kg, 0.02 to 0.25 mg/kg, 0.02 to 0.15 mg/kg and 0.02 to 0.07 mg/kg given as a single once a day dose or as single or divided doses throughout the day.

The compound of formula (I) or a salt or solvate thereof may be employed in the treatment of anxiety disorders or in the treatment of mixed anxiety-depressive disorder as the compound per se, but is preferably presented with one or more pharmaceutically acceptable carriers, diluents or excipients in the form of a pharmaceutical formulation. The carriers, diluents and exipients must, of course, be acceptable in the sense of being compatible with the other ingredients of the formulation and must not be deleterious to the recipient. The carrier may be a solid or a liquid, or both, and is preferably formulated with the agent as a unit-dose formulation, for example, a tablet containing 1 mg, 2 mg, 5 mg, 10 mg, 20 mg, 40 mg, 60 mg, 80 mg, 100 mg, 120 mg, 150 mg and 200 mg of the compound of formula (I) or a salt or solvate thereof, more preferably 10-80 mg or 20-160 mg, of the compound of formula (I) or a salt or solvate thereof. Suitable formulations for use in the present invention include sustained release solid-dosage formulations, optionally film-coated solid-dosage formulations, and especially tablet and caplet formulations, for oral administration of the compound of formula (I), particularly once-daily administration, for example those illustrated in Examples 1 to 5 below.

The formulations include those suitable for oral, rectal, topical, buccal (e.g. sub-lingual) and parenteral (e.g. subcutaneous, intramuscular, intradermal or intravenous) administration.

Formulations suitable for buccal (sub-lingual) administration include lozenges comprising a compound of formula (I) or a salt or solvate thereof in a flavoured base, usually sucrose and acacia or tragacanth, and pastilles comprising the agent in an inert base such as gelatin and glycerin or sucrose and acacia.

Formulations of the present invention suitable for parenteral administration conveniently comprise sterile aqueous preparations of a compound of formula (I) or a salt or solvate thereof, preferably isotonic with the blood of the intended recipient. These preparations are preferably administered intravenously, although administration may also be effected by means of subcutaneous, intramuscular, or intradermal injection. Such preparations may conveniently be prepared by admixing the agent with water and rendering the resulting solution sterile and isotonic with the blood.

Formulations suitable for rectal administration are preferably presented as unit-dose suppositories. These may be prepared by admixing a compound of formula (I) or a salt or solvate thereof with one or more conventional solid carriers, for example, cocoa butter, and then shaping the resulting mixture.

Formulations suitable for topical application to the skin preferably take the form of an ointment, cream, lotion, paste, gel, spray, transdermal patch, aerosol, or oil. Carriers which may be used include vaseline, lanolin, polyethylene glycols, alcohols, and combinations of two or more thereof.

It should be understood that in addition to the ingredients particularly mentioned above, the formulations may include other agents conventional in the art having regard to the type of formulation in question.

### FORMULATION EXAMPLES

The following non-limiting examples illustrate suitable formulations for use in the present invention, particularly for once-daily administration.

### Example 1

| Component | Amount/unit (mg) |
|---|---|
| Tablet Core | |
| Formula (I). HCl | 22.86(*) |
| Microcrystalline Cellulose (Avicel PH102) | 176.20 |
| Ph. Eur/USNF | |
| Hydroxypropylmethylcellulose (Methocel E4M CR) | 120.25 |
| Ph. Eur/USNF | |
| Sodium Bisulphate | 3.25 |
| Purified Water (removed during processing) | qs |
| Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 20 mg of the compound of formula (I)

### Example 2

| Component | Amount/unit (mg) |
|---|---|
| Tablet Core | |
| Formula (I). HCl | 45.72(*) |
| Microcrystalline Cellulose (Avicel PH102) | 159.84 |
| Ph. Eur/USNF | |
| Hydroxypropylmethylcellulose (Methocel E4M CR) | 113.75 |
| Ph. Eur/USNF | |
| Sodium Bisulphate | 3.25 |
| Purified Water (removed during processing) | qs |
| Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 40 mg of the compound of formula (I)

### Example 3

| Component | Amount/unit (mg) |
|---|---|
| Tablet Core | |
| Formula (I). HCl | 68.58(*) |
| Microcrystalline Cellulose (Avicel PH102) | 153.23 |
| Ph. Eur/USNF | |
| Hydroxypropylmethylcellulose (Methocel E4M CR) | 97.50 |
| Ph. Eur/USNF | |
| Sodium Bisulphate | 3.25 |
| Purified Water (removed during processing) | qs |
| Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 60 mg of the compound of formula (I)

### Example 4

| Component | Amount/unit (mg) |
|---|---|
| Tablet Core | |
| Formula (I). HCl | 91.44(*) |
| Microcrystalline Cellulose (Avicel PH102) | 130.37 |
| Ph. Eur/USNF | |
| Hydroxypropylmethylcellulose (Methocel E4M CR) | 97.50 |
| Ph. Eur/USNF | |
| Sodium Bisulphate | 3.25 |
| Purified Water (removed during processing) | qs |
| Ph. Eur | |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 80 mg of the compound of formula (I)

### Example 5

| Component | Amount/unit (mg) |
|---|---|
| Tablet Core | |
| Formula (I). HCl | 11.43(*) |
| Microcrystalline Cellulose (Avicel PH102) | 160.87 |
| Ph. Eur/USNF | |
| Hydroxypropylmethylcellulose (Methocel E4M CR) | 146.25 |
| Ph. Eur/USNF | |
| Sodium Bisulphate | 3.25 |
| Purified Water (removed during processing) | qs |
| Ph. Eur | |
| Magnesium Stearate Ph. Eur/USNF | 3.25 |
| TOTAL | 325.05 |
| Tablet coating | |
| Opadry White: YS-1R-7003 | 9.75 |
| TOTAL | 334.80 |

(*)corresponding to 10 mg of the compound of formula (I)

Examples 1 to 5 above were prepared by a process similar to the following general process: The drug substance is blended and wet granulated with the pharmaceutically acceptable excipients described, including HPMC as the rate-controlling polymer. The acidic stabiliser (sodium bisulphate) is first dissolved in purified water to produce the granulation solution, and the granule is then produced by conventional processing techniques, for example either high shear or a fluid bed process, followed by drying, milling, blending, compression into a tablet, and finally aqueous film-coating.

### Biological Data

In vitro Synaptosomal Uptake

In vitro uptake was determined, as reported previously, using synaptosomes prepared from rat caudoputamen (for dopamine uptake) and hypothalamus (for NA and serotonin uptake) using [³H]-dopamine, [³H]-NA and [³H]-serotonin as transport substrates, respectively. See Eckhardt, S. B., R. A. Maxwell, and R. M. Ferris, A Structure-Activity Study of the Transport Sites for the Hypothalamic and Striatal Catecholamine Uptake Systems. Similarities and differences. Molecular Pharmacology, 21: p. 374-9, 1982.

Synaptosomes for use in obtaining in vitro uptake data were prepared from hypothalamus or striatum by gently homogenizing the tissue in a 0.3 M sucrose/25 mM Tris pH 7.4 buffer containing iproniazid phosphate to inhibit monoamine oxidase. The homogenate was centrifuged at 1100×g at 4° C. for 10 min and the supernatant was used for uptake studies. The supernatant (˜1 mg tissue protein) was incubated with Km concentrations of [³H]-noradrenaline, [³H]-dopamine or [³H]-serotonin at 37° C. for 5 minutes in Modified Krebs-Henseleit buffer (118 mM NaCl, 5 mM KCl, 25 mM NaHCO₃, 1.2 mM NaH₂PO₄, 1.2 mM MgSO₄, 11 mM Dextrose, 2.5 mM CaCl₂) in the absence and presence of drug. Under these conditions uptake was linear with respect to both for substrate and tissue (with <5% total substrate transported). Non-specific uptake was defined as uptake at 0° C. [³H]-substrate, which had been transported into synaptosomes, was separated from free [³H]-substrate by filtration over GF/B filters and washing with cold Krebs-Henseleit buffer. The filters were counted for tritum in a liquid scintillation spectrometer.

The data for in vitro synaptosomal uptake are presented below as Table 1. The compound of formula (I), inhibited noradrenaline (NA) uptake with an IC₅₀ of 1.1 μM. On dopamine (DA) uptake, the compound of formula (I) had an IC₅₀ of ˜10 μM. The compound of formula (I) showed no inhibition of serotonin uptake at 30 μM.

TABLE 1

| Compound | IC₅₀ NA | IC₅₀ DA | IC₅₀ Serotonin |
|---|---|---|---|
| Formula (I) | 1.1 ± 0.07 | 9.3 ± 0.41 | >30 |

Uptake values are means ± SEM of 3 separate experiments. The IC₅₀ values are concentrations (μM) required for 50% inhibition of uptake.

Functional Reuptake Inhibition on Human Monoamine Transporters

Three separate cell-lines expressing human monoamine transporters for dopamine (hDAT) noradrenaline (hNET) and serotonin (hSERT) were used to measure the functional reuptake inhibiting properties of the compound of formula (I) (as its hydrochloride salt). The following methods were utilised.

Human noradrenaline transporter (hNET): MDCK/hNET (dog kidney) cells (4×10⁴ cells/well) expressing the human norepinephrine transporter were plated on 96-well format one day before the assay. When the cells were 80% confluent, cell monolayers were washed and preincubated with test compound and/or vehicle in modified Tris-HEPES buffer pH 7.1 at 25° C. for 20 minutes, then 25 nM [³H]Norepinephrine was added to make the total volume to 200 μl and the cells were further incubated for 10 minutes. Cells in the well were then rinsed twice, solubilized with 1% SDS lysis buffer and the lysate was counted to determine [³H]Norepinephrine uptake. Non-specific signal was determined in the presence of 10 μM desipramine. Reduction of [³H]Norepinephrine uptake by 50 percent or more (≧50%) relative to vehicle controls indicated significant inhibitory activity.

Human dopamine transporter (hDAT): CHO-K1/hDAT cells (8×10⁴ cells/well) expressing the human dopamine transporter (hDAT) were plated on 96-well format one day before the assay. Cells were preincubated with test compound and/or vehicle in modified Tris-HEPES buffer pH 7.1 at 25° C. for 20 minutes, then 50 nM [³H]Dopamine was added to make the total volume to 200 μl and further incubated for 10 minutes. Cells in the well were then rinsed twice, solubilized with 1% SDS lysis buffer and the lysate was counted to determine [³H]Dopamine uptake. Non-specific signal was determined in the presence of 10 μM nomifensine. Reduction of [³H]Dopamine uptake by 50 percent or more (≧50%) relative to vehicle controls indicates significant inhibitory activity.

Human serotonin transporter (hSERT): HEK-293/hSERT cells (5×10⁴ cells/tube) expressing the human serotonin transporter (hSERT) were added into the minitube on 96-tube holder prior to assay. Cells were preincubated with test compound or vehicle in modified Tris-HEPES buffer pH 7.1 at 25° C. for 20 minutes, then 65 nM [³H]Serotonin was added to make the total volume to 200 μl and further incubated for 10 minutes. Cells were then washed by filtration through cell harvester four times with PBS buffer containing 0.1% BSA and the GF/B filter was counted to determine [³H]Serotonin uptake. Non-specific signal was determined in the presence of 10 μM fluoxetine. Reduction of [³H]Serotonin uptake by 50 percent or more (≧50%) relative to vehicle-control indicates significant inhibitory activity.

Compounds were screened at 10, 1, 0.1, 0.01 and 0.001 μM. These same concentrations were concurrently applied to a separate group of untreated cells and evaluated for possible compound-induced cytotoxicity only if significant inhibition of uptake was observed. Radioactivity retained on the filters was determined by scintillation counting overnight using a Packard scintillation counter.

The potencies for monoamine reuptake inhibition for the hydrochloride salt of the compound of formula (I) are expressed in Table 2 below as IC₅₀ (in μM; mean±SEM) following three separate experiments, each performed in duplicate (n=3). The compound demonstrated reuptake inhibition at both hDAT (pIC50=6.36) and hNET (pIC50=6.70) but reuptake inhibition was not observed on hSERT (pIC50<5) at the highest concentration tested (10 μM). No cytotoxicity was observed at any of the concentrations causing reuptake inhibition.

TABLE 2

| Compound | hNET | hDAT | hSERT |
|---|---|---|---|
| Formula (I). HCl | 0.20 ± 0.05 | 0.44 ± 0.01 | >10 |
| | (n = 3) | (n = 3) | (n = 3) |

The compound of formula (I) was also tested in well-characterised models of anxiety and was found to be active as follows.

Social Interaction Test in Rats
[Animal Models for Predicting Clinical Efficacy of Anxiolytic Drugs: Social Behaviour. Neuropsychobiology. 13 (1-2) 55-62, 1985]

Male Sprague Dawley rats (350-400 g at day of test, Charles River, Italy) were individually housed for five days before the test session. The 15 minutes social interaction test was performed between 9:00 to 14:00. Pairs of rats, weight and treatment matched, were placed in the test arena under high light condition. Active social interaction (sniffing, following, grooming, biting, boxing, crawling over and under) and motor activity, in terms of line crossing were scored by an observer blind to the drug treatment. The animals were treated orally with vehicle (2 ml/kg) or with the hydrochloride salt of the compound of formula (I) at 3, 10 and 30 mg/kg, 45 minutes prior to the test (acute study), or for 20 days twice a day and with a final treatment given on day 21, the last treatment given 45 minutes before the test (chronic study). An additional group of rats (positive control group) received a single oral dose of chlordiazepoxide (CDP) 4 mg/kg 1 hour before testing.

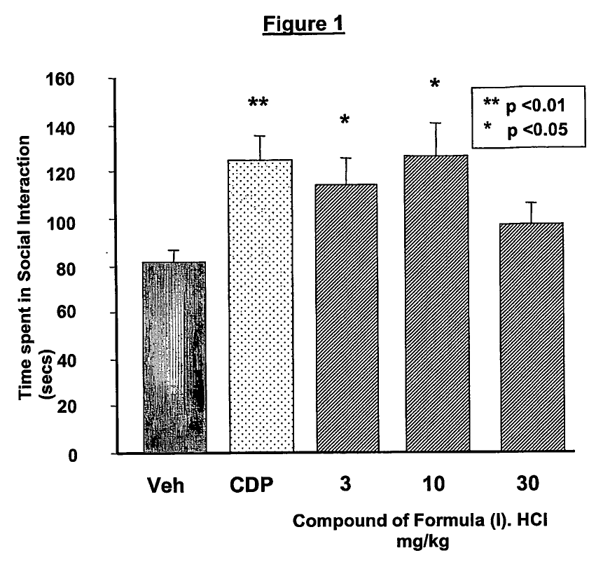

The results of the chronic study are given below in Table 3 (values given as average±s.e.), with the data for social interaction also illustrated in FIG. 1.

TABLE 3

| | | Compound of | Compound of | Compound of | |
|---|---|---|---|---|---|
| | | Formula (I) | Formula (I) | Formula (I) | |
| | | HCl salt | HCl salt | HCl salt | CDP |
| | Vehicle | 3 mg/kg | 10 mg/kg | 30 mg/kg | 4 mg/kg |
| | (n = 14) | (n = 14) | (n = 10) | (n = 14) | (n = 14) |
| Time spent in active | 81.2 ± 5.6 s | 114.0 ± 11.5 s* | 126.4 ± 13.8 s* | 97.6 ± 8.7 s | 124.4 ± 10.7 s** |
| social interaction | | 40.4% | 55.7% | 20.2% | 53.2% |
| Number of line crossings | 603.7 ± 17.6 | 587.0 ± 29.1 | 571.0 ± 44.1 | 476.2 ± 42.6** | 560.1 ± 29.6 |

*p < 0.05
**p < 0.01;
ANOVA, Dunnett's test

The hydrochloride salt of the compound of formula (I) was able to induce an anxiolytic-like effect in this test at 3 and 10 mg/kg without any significant effects on locomotor activity; at 30 mg/kg a significant reduction in locomotor activity compared to vehicle treated animals was observed, which may explain the lack of effect on social interaction at this dose.

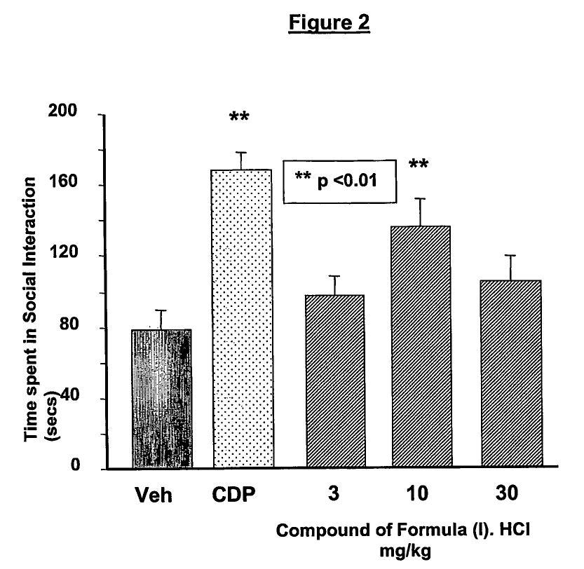

The results for social interaction in the acute study are illustrated in FIG. 2.

Light & Dark Box Test in Mice

[Costall B. Jones B J. Kelly M E. Naylor R J. and Tomkins D M. Exploration of Mice in a Black and White Test Box: Validation as a Model of Anxiety. Pharmacology, Biochemistry & Behavior. 32 (3):777-85, 1989]

The light-dark box test consists of a Perspex box which is divided into two compartments, two-thirds of which is painted white and the remaining third is painted black. Mice are able to move freely between the two compartments using a small gap in the wall separating the two compartments. The floor is marked with lines, which provided an index of motor activity of the animals (number of line crossings). A white light (60 W) is placed over the white compartment of the box to provide a brightly-lit area which the animals find aversive. Mice tend to escape from this area preferring to remain in the black area, returning only occasionally to explore the white area. Anxiolytic compounds increase, dose dependently, the time the animals spend in the aversive white area.

CD-1 mice (25-30 g, Charles River, Italy) were placed individually into the centre of the white side of the black/white test box for the 5 min test period and the behaviour of the mice was recorded by remote video recording. Behaviour of the mice was assessed subsequently from the tape recordings by an observer who was unaware of the animals' treatment. Different parameters were assessed to estimate both the anxiolytic and the sedative effects of the compound tested. These include the time spent in the white compartment, latency to the initial movement, number of line crossings in the white and the black compartment, number of rearings in the white and the black compartment, and number of transitions between the two compartments.

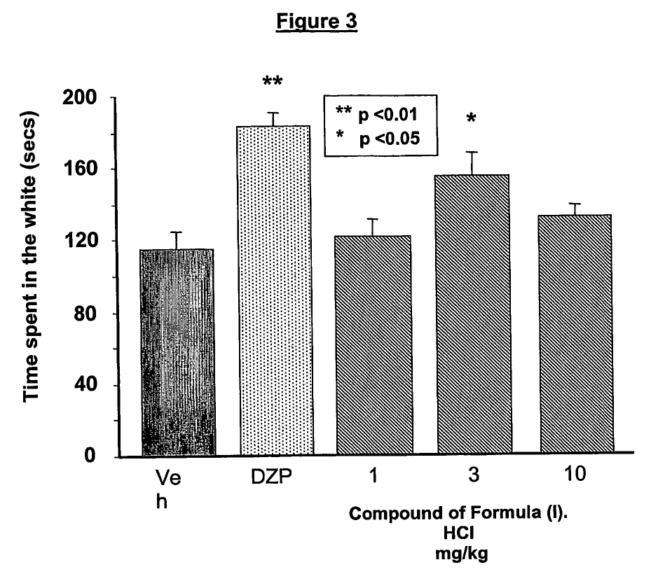

The animals were orally treated (10 ml/kg) with the hydrochloride salt of the compound of formula (I) at 1, 3 and 10 mg/kg, 30 minutes before test. An additional group of rats (positive control group) received an oral dose of diazepam (DZP) 1 mg/kg 1 hour before testing. The results are given in FIG. 3, which demonstrate that the hydrochloride salt of the compound of formula (I) was able to induce an anxiolytic-like effect in this test at 3 mg/kg.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1-4. (canceled)

5. A method of treating anxiety disorders in a mammalian subject comprising the administration to said subject of an effective amount of (+)-(2S,3S)-2-(3-chlorophenyl)-3,5,5-trimethyl-2-morpholinol or a salt or solvate thereof or pharmaceutical compositions thereof.

6. A method of treating mixed anxiety-depressive disorder in a mammalian subject comprising the administration to said subject of an effective amount of (+)-(2S,3S)-2-(3-chlorophenyl)-3,5,5-trimethyl-2-morpholinol or a salt or solvate thereof or pharmaceutical compositions thereof.

7. The method according to claim 5 wherein said (+)-(2S,3S)-2-(3-chlorophenyl)-3,5,5-trimethyl-2-morpholinol or the salt or solvate thereof is enantiomerically pure.

8. The method as claimed in claim 5 wherein the salt is (+)-(2S,3S)-2-(3-chlorophenyl)-3,5,5-trimethyl-2-morpholinol hydrochloride.

9. The method according to claim 6 wherein said (+)-(2S,3S)-2-(3-chlorophenyl)-3,5,5-trimethyl-2-morpholinol or the salt or solvate thereof is enantiomerically pure.

10. The method as claimed in claim 6 wherein the salt is (+)-(2S,3S)-2-(3-chlorophenyl)-3,5,5-trimethyl-2-morpholinol hydrochloride.
